(19) United States
(12) Patent Application Publication
(10) Pub. No.: US 2018/0185502 A1
(43) Pub. Date: Jul. 5, 2018

(54) IMMOBILIZATION OF BIOMOLECULES BY SELF-ASSEMBLED NANOSTRUCTURES

(71) Applicant: OHIO STATE INNOVATION FOUNDATION, Columbus, OH (US)

(72) Inventors: Jon Robert PARQUETTE, Hilliard, OH (US); Sriram SATAGOPAN, Columbus, OH (US); Yuan SUN, Columbus, OH (US); Fred Robert TABITA, Dublin, OH (US)

(21) Appl. No.: 15/739,375

(22) PCT Filed: Jun. 23, 2016
(86) PCT No.: PCT/US16/39034

§ 371 Date: Dec. 22, 2017

Related U.S. Application Data
(60) Provisional application No. 62/183,560, filed on Jun. 23, 2015.

(51) Int. Cl. A61K 47/64; A61K 47/54; C21B 13/00; A61K 47/69; B01D 53/62; B01D 53/86; B01D 53/88

(52) U.S. Cl. CPC A61K 47/64; A61K 47/542; C21B 13/0073; A61K 47/6925; B01D 53/62; B01D 53/8671; C12Y 402/01001; B01D 2255/804; B01D 2255/90; B01D 2257/504; B01D 2258/06; C12Y 401/01039; B01D 53/88

(57) ABSTRACT

Disclosed are nanostructures such as carboxysomes that encapsulate RubisCO and carbonic anhydrase to provide a protected environment to maximize CO₂ assimilation. Conditions are disclosed were RubisCO can be sequestered into a variety of self-assembling nanotubes. The encapsulated protein was enzymatically active and was clearly associated with the nanotubes and removed from solution based on a number of criteria. These nanostructures were also found to enhance the stability of RubisCO toward proteases and other environmental factors. These structures can be used in scalable CO₂ conversions and other processes.

### CROSS-REFERENCE TO RELATED APPLICATIONS

This application claims the benefit of priority to U.S. Provisional Application No. 62/183,560, filed Jun. 23, 2015, which is hereby incorporated herein by reference in its entirety.

### BACKGROUND

Carbon dioxide (CO₂) is an abundant greenhouse gas, trapping thermal radiation close to the earth's atmosphere and contributing to global warming and climate change. CO₂ emissions are expected to increase by more than 40% by 2035, unless major worldwide policies are soon implemented. From an industrial perspective, CO₂ represents a large source of carbon for the synthesis of a large range of chemicals. While plants and microbes are efficient at converting CO₂ into sugars and other compounds, the sophisticated chains of enzymatic reactions that usually accomplish these processes are difficult to replicate in an industrial context. Catalysis represents over 90% of the chemical processes currently utilized by industry, with an annual market value of over US $1 trillion. Much of the feedstock for industrial catalytic processes continues to be petroleum-based, making this endeavor not ideal from a sustainability perspective. Strategies to sequester and convert CO₂ into usable feedstocks have become increasingly important to slow global warming and reduce dependence on fossil fuels. Although biological systems are among the most efficient and ubiquitous catalysts for CO₂ fixation, the use of free or cell-based enzymes as biocatalysts for large-scale industrial processes pose significant drawbacks due to their incompatibility with reaction conditions that often depart from their physiological states. The challenge is to construct catalytic systems that mimic the cellular environment but are scalable and sufficiently robust to withstand harsher conditions and be separated from the product. The subject matter disclosed herein addresses these and other needs.

### SUMMARY

In accordance with the purposes of the disclosed materials, compounds, compositions, articles, devices, and methods, as embodied and broadly described herein, the disclosed subject matter relates to compositions and methods of making and using the compositions. In a specific aspect, disclosed are self-assembled nanotubes that comprise a wall, wherein the wall is formed from a conjugate. The conjugate can comprise a hydrophobic compound linked to a hydrophilic amino acid or peptide and can self-assemble into the nanotube wall. The compositions also comprise an enzyme sequestered in (encapsulated by) the nanotube. Methods for forming the conjugates, nanotubes, and compositions, and using them to stabilize the enzymes are also disclosed.

Also, disclosed are the encapsulation/immobilization of RubisCO, a CO₂-fixing enzyme, by self-assembled nanotubes and other nanostrutures. These nanostructures enhance the stability of RubisCO toward proteases and other environmental factors that permit these biocatalysts to be useful in scalable CO₂ conversion processes. The approach described for RubisCO can be applicable to other biomolecules including other enzymes, cytokines and other biomolecules such as RNA and DNA. This invention can contribute to the production of biofuels and bioproducts from sources other than petroleum. The nanostructure-RubisCO construct described herein can facilitate the identification of the optimal catalytic platform for greenhouse gas conversion with regard to catalyst robustness, kinetic efficiency and recyclability.

Additional advantages of the disclosed subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplar) and explanatory only and are not restrictive.

### BRIEF DESCRIPTION OF THE FIGURES

The accompanying Figures, which are incorporated in and constitute a part of this specification, illustrate several aspects of the invention and together with the description serve to explain the principles of the invention.

FIG. 1 displays dimeric Rhodospirillum rubrum RubisCO (shadded ribbons) catalyzes the addition of CO₂ to RuBP (black) resulting in two molecules of 3-phosphoglycerate, which are utilized by the host organisms (primary producers) to produce usable energy-rich sugars and to regenerate RuBP.

FIG. 2 (top) displays Self-Assembly of lysine NDI bolaamphile into nanotubes. FIG. 2 (bottom) displays Nanotubes formed CPT-dipeptides A (Ac-KK(CPT) and B (NH₂-KK(CPT).

FIG. 3 displays TEM images obtained without stain to enhance visualization of RubisCO-nanotube assembly. Nanotubes formed in Tris-Cl buffer from CPT-dipeptides A (Ac-KK(CPT) and B (NH₂-KK(CPT), alone (FIG. 3A-3B) or in the presence of 0.1 mg/mL RubisCO dimer (FIG. 3C-3D).

FIG. 4A displays 5-nm Ni-NTA-NANOGOLD™ particles (Nanoprobes, Inc.) used to target the histidine-tagged R. rubrum RubisCO for easy visualization in TEM. Also in FIG. 4A is a notional depiction of Nanogold tagging of R. Rubrum RubisCO. FIG. 4B displays TEM images of histidine-tagged R. rubrum RubisCO bound to nanotubes formed by CPT-dipeptide A (Ac-KK(CPT). FIGS. 4C-4D are TEM images of histidine-tagged R. rubrum RubisCO bound to nanotubes formed by CPT-dipeptide B (NH₂-KK(CPT). The dark dots decorating the nanotubes along the inner and other wall surfaces represent bound RubisCO.

FIG. 5 is a plot showing activity of R. Rubrum RubisCO with or without nanotube in presence of proteolytic enzyme subtilisin.

### DETAILED DESCRIPTION

The compounds, compositions, articles, devices, and methods described herein can be understood more readily by reference to the following detailed description of specific aspects of the disclosed subject matter and the Examples and Figures.

Before the present compounds, compositions, articles, devices, and methods are disclosed and described it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

General Definitions

In this specification and in the claims that follow, reference will be made to a number of terms, which shall be defined to have the following meanings:

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

When ranges of values are disclosed, and the notation “from n₁ . . . to n₂” is used, where n₁ and n₂ are the numbers, then unless otherwise specified, this notation is intended to include the numbers themselves and the range between them. This range can be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 M, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

Chemical Definitions

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

When substituted, the substituents of a substituted group can include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen or deuterium, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents can be joined together to form a fused five-, six-, or seven-membered carbocyclic or heterocyclic ring consisting of zero to three heteroatoms, for example forming methylenedioxy or ethylenedioxy. An optionally substituted group can be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O, which is also referred to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “lower,” as used herein, alone or in a combination, where not otherwise specifically defined, means containing from 1 to and including 6 carbon atoms.

The term “lower alkyl,” as used herein, alone or in a combination, means C₁-C₆ straight or branched chain alkyl. The term “lower alkenyl” means C₂-C₆ straight or branched chain alkenyl. The term “lower alkynyl” means C₂-C₆ straight or branched chain alkynyl.

The term “lower aryl,” as used herein, alone or in combination, means phenyl or naphthyl, either of which can be optionally substituted as provided.

The term “lower heteroaryl,” as used herein, alone or in combination, means either 1) monocyclic heteroaryl comprising five or six ring members, of which between one and four said members can be heteroatoms chosen from O, S, and N, or 2) bicyclic heteroaryl, wherein each of the fused rings comprises five or six ring members, comprising between them one to four heteroatoms chosen from O, S, and N.

The term “lower cycloalkyl,” as used herein, alone or in combination, means a monocyclic cycloalkyl having between three and six ring members. Lower cycloalkyls can be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four can be heteroatoms chosen from O, S, and N. Examples of lower heterocycloalkyls include pyrrolidinyl, imidazolidinyl, pyrazolidinyl, piperidinyl, piperazinyl, and morpholinyl. Lower heterocycloalkyls can be unsaturated.

The term “lower carboxyl,” as used herein, alone or in combination, means —C(O)R, wherein R is chosen from hydrogen, lower alkyl, cycloalkyl, cycloheterolkyl, and lower heteroalkyl, any of which can be optionally substituted with hydroxyl, (O), and halogen.

The term “lower amino,” as used herein, alone or in combination, refers to —NRR′, wherein R and R′ are independently chosen from hydrogen, lower alkyl, and lower heteroalkyl, any of which can be optionally substituted. Additionally, the R and R′ of a lower amino group can combine to form a five- or six-membered heterocycloalkyl, either of which can be optionally substituted.

The term “nitro” as used herein is represented by the formula —NO₂.

The term “nanotube” is used herein in a general sence to refer to an elongated nanostructure. This term is meant to include nanobars, nanowhiskers, helixes, nanospheres, and the like. In some examples, the nanotube is not a β-sheet.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “peptide” as used herein refers to short polymers formed from the linking, in a defined order, of α-amino acids. The link between one amino acid residue and the next is known as an amide bond or a peptide bond. Proteins are polypeptide molecules. The distinction is that peptides are short and polypeptides/proteins are long. There are several different conventions to determine these. Peptide chains that are short enough to be made synthetically from the constituent amino acids are called peptides, rather than proteins, with one dividing line at about 50 amino acids in length.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

Disclosed herein are compositions that comprising: a self-assembled nanotube comprising a conjugate comprising hydrophobic compound, a hydrophilic amino acid residue or peptide; and an optional linker moiety joining the hydrophobic compound to the hydrophilic amino acid or peptide, wherein the conjugate forms a self-assembled nanotube, and an enzyme, wherein the enzyme is sequestered in the self-assembled nanotube. Each of these components is discussed in more detail below.

### Nanotubes

In the disclosed compositions there are self-assembled nanotubes comprising a conjugate. Disclosed herein are conjugates that comprise a hydrophobic compound linked via a linker moiety to a protected or unprotected peptide or single amino acid. The conjugates can self-assemble into nanotubes so that the walls of the nanotubes are characterized by a hydrophilic domain comprising the peptide component of the conjugate and a hydrophobic domain comprising the hydrophobic compound. By sequestering the enzyme within the nanotube walls, the enzyme can be protected and stabilized.

Thus, disclosed herein is a nanotube having a wall, wherein the wall comprises a hydrophobic domain and a hydrophilic domain, and wherein the hydrophobic domain comprises a hydrophobic compound and the hydrophilic domain comprises an amino acid or peptide. The general structure of a nanotube wall as disclosed herein can be shown as follows:

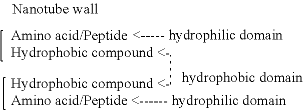

In this wall schematic there are two conjugates shown, each comprising an amino acid or peptide linked to a hydrophobic compound. The conjugates are thus amphiphilic with a hydrophilic portion comprising the amino acid or peptide and a hydrophobic portion comprising the compound. In the simplest sense, two conjugates assemble such that the hydrophobic compound portion of each conjugate associate together and create the internal, hydrophobic domain of the wall, and the amino acid or peptide portion of each conjugate is directed outward and create the hydrophilic domain of the wall. This arrangement is repeated linearly many times over to create the wall of the disclosed nanotube. It is also contemplated that the disclosed nanotubes can be single walled as shown above, or double-walled where one wall is on top of the other. It is also contemplated that the disclosed nanotubes can have more than two walls.

The disclosed nanotube can be defined by its aspect ratio, which is the length of the nanotube divided by the width of the nanotube. The disclosed nanotube can have an aspect ratio of at least about 5; for example, the nanotube can have an aspect ratio of at least about 10, at least about 15, at least about 20, or at least about 25. In some examples, the disclosed nanotube can have an aspect ratio that is about 25 or less; for example, the nanotube can have an aspect ratio of about 20 or less, about 15 or less, about 10 or less, or about 5 or less). The disclosed nanotube can have an aspect ratio ranging from any of the minimum values described above to any of the maximum values described above. For example, the nanotube can have an aspect ratio ranging from about 5 to about 25 (e.g., from at least about 10 to about 20, from about 15 to about 25, from about 10 to about 15, or from about 20 to about 25).

In certain examples, the disclosed nanotube can have a length ranging from about 1 nm to about 500 nm. In specific examples, the disclosed nanotube can have a length ranging from about 1 nm to about 400 nm, from about 1 nm to about 300 nm, from about 1 nm to about 200 nm, from about 1 nm to about 100 nm, from about 100 nm to about 500 nm, from about 100 nm to about 400 nm, from about 100 nm to about 300 nm, from about 100 nm to about 200 nm, from about 200 nm to about 500 nm, from about 200 nm to about 400 nm, from about 200 nm to about 300 nm, from about 300 nm to about 500 nm, from about 300 nm to about 400 nm, or from about 400 nm to about 500 nm. In other examples, the nanotube can have a length of greater than about 500 nm. For examples, the nanotube can have a length ranging from about 500 to about 5 μm, from about 1 μm to about 4 μm, from about 1 μm to about 3 μm, from about 1 to about 2 μm, from about 2 μm to about 5 μm, from about 2 μm to about 4 μm, from about 2 μm to about 3 μm, from about 3 μm to about 5 μm, from about 3 μm to about 4 μm, or from about 4 μm to about 5 μm. It is also contemplated that the disclosed nanotube can have a length of greater than 5 μm.

The surface charge of the disclosed nanotube can influence the stability and movement of the nanotube in tissue. The disclosed nanotube can have a negative Zeta potential, which enhances cell penetration but lowers in vivo stability and mobility. It has been found that near-zero Zeta potentials are preferred, though positive Zeta potential can also be used. For example, the disclosed nanotube can have a Zeta potential of from about −50 mV to about +50 mV, from about −40 mV to about +40 mV, from about −30 mV to about +30 mV, from about −20 mV to about +20 mV, from about −10 mV to about +10 mV, from about −5 mV to about +5 mV, from about −1 mV to about +1 mV. In a preferred example, the disclosed nanotube can have a Zeta potential of about 0 mV.

### Conjugates

As mentioned herein, the disclosed nanotube can have one or more walls, each made from conjugates that contain a hydrophobic compound linked to an amino acid or peptide. Thus, in another aspect, disclosed herein is such a conjugate, which can be represented by Formula I.

D-L-AA (I)

where D is the hydrophobic compound, L is a linker moiety, and AA is an amino acid residue of a single amino acid or a peptide. In a specific example, the hydrophobic compound is NDI.

### Amino Acid or Peptide (AA)

In the disclosed conjugate, the hydrophobic compound is linked to a single amino acid residue or an amino acid residue of a peptide. This component is shown as AA in Formula I. The particular amino acid or peptide should be hydrophilic so that the conjugate will self assemble in aqueous environments into the nanotube wall. When using a peptide, one or more amino acid residues in the peptide can be hydrophobic or neutral, as long as the overall peptide component is hydrophilic.

The amino acids in Table 1 can be present as residues in the peptide component of the disclosed conjugates.

TABLE 1
Amino Acid Abbreviations

| Amino Acid | Abbreviations |
|---|---|
| alanine | Ala (A) |
| allosoleucine | AIle |
| arginine | Arg (R) |
| asparagine | Asn (N) |
| aspartic acid | Asp (D) |
| cysteine | Cys (C) |
| glutamic acid | Glu (E) |
| glutamine | Gln (K) |
| glycine | Gly (G) |
| histidine | His (H) |
| isolelucine | Ile (I) |
| leucine | Leu (L) |
| lysine | Lys (K) |
| phenylalanine | Phe (F) |
| methionine | Met (M) |
| proline | Pro (P) |
| pyroglutamic acid | PGlu |
| serine | Ser (S} |
| threonine | Thr (T) |
| tyrosine | Tyr (Y) |
| tryptophan | Trp (W) |
| valine | Val (V) |

When a single amino acid residue is present in the conjugate, the preferred residues are arginyl, histidyl, lysyl, aspartyl, glutamyl, seryl, threonyl, cystyl, asparagyl, glutaminyl, prolyl, tyrosyl, methionyl, and tryptophanyl. These moieties can be attached to the hydrophobic by a linker at the amino group, the carboxylate group, or the side chain. In certain, examples, the amino acid residue is a lysyl.

When two amino acid residues are present in the conjugate and they are coupled by a peptide bond, the resulting dipeptide can contain any of the residues in Table 1 as long as the overall dipetide is hydrophilic. For example, the dipeptide can comprise two arginyls, histidyls, lysyls, aspartyls, glutamyls, seryls, threonyls, cystyls, asparagyls, glutaminyls, prolyls, tyrosyls, methionyls, or tryptophanyls. In other examples the dipeptide comprises at least one of arginyl, histidyl, lysyl, aspartyl, glutamyl, seryl, threonyl, cystyl, asparagyl, glutaminyl, prolyl, tyrosyl, methionyl, or tryptophanyl.

In other examples, the didpetide can comprise arginyl with alanyl, allosoleucyl, asparagyl, aspartyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

In other examples, the didpetide can comprise histidyl with alanyl, allosoleucyl, arginyl, asparagyl, aspartyl, cystyl, glutamyl, glutaminyl, glycyl, isolelucyl, leucyl, lysyl, methionyl, phenylalanyl, prolyl, pyroglutamyl, seryl, threonyl, tyrosyl, tryptophanyl, or valyl.

In other examples, the didpetide can comprise lysyl with alanyl, allosoleucyl, arginyl, asparagyl, aspartyl, cystyl, glutamyl, glutaminyl, glycyl, histidyl, isolelucyl, leucyl, methionyl, phenylalanyl, prolyl, pyroglutamyl, seryl, threonyl, tyrosyl, tryptophanyl, or valyl.

In other examples, the didpetide can comprise aspartyl with alanyl, allosoleucyl, arginyl, asparagyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

In other examples, the didpetide can comprise glutamyl with alanyl, allosoleucyl, arginyl, asparagyl, aspartyl, cystyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

In other examples, the didpetide can comprise seryl with alanyl, allosoleucyl, arginyl, asparagyl, aspartyl, cystyl, glutamyl, glutaminyl, glycyl, histidyl, isolelucyl, leucyl, lysyl, methionyl, phenylalanyl, prolyl, pyroglutamyl, threonyl, tyrosyl, tryptophanyl, or valyl.

In other examples, the didpetide can comprise threonyl with alanyl, allosoleucyl, arginyl, asparagyl, aspartyl, cystyl, glutamyl, glutaminyl, glycyl, histidyl, isolelucyl, leucyl, lysyl, methionyl, phenylalanyl, prolyl, pyroglutamyl, seryl, tyrosyl, tryptophanyl, or valyl.

In other examples, the didpetide can comprise cystyl with alanyl, allosoleucyl, arginyl, asparagyl, aspartyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

In other examples, the didpetide can comprise asparagyl with alanyl, allosoleucyl, arginyl, aspartyl, glutamyl, glutaminyl, glycyl, histidyl, isolelucyl, leucyl, lysyl, methionyl, phenylalanyl, prolyl, pyroglutamyl, seryl, cystyl threonyl, tyrosyl, tryptophanyl, or valyl.

In other examples, the didpetide can comprise glutaminyl with alanyl, allosoleucyl, arginyl, asparagyl, aspartyl, glutamyl, glycyl, histidyl, isolelucyl, leucyl, lysyl, methionyl, phenylalanyl, prolyl, pyroglutamyl, seryl, cystyl threonyl, tyrosyl, tryptophanyl, or valyl.

In other examples, the didpetide can comprise prolyl with alanyl, allosoleucyl, arginyl, asparagyl, aspartyl, glutamyl, glutaminyl, glycyl, histidyl, isolelucyl, leucyl, lysyl, methionyl, phenylalanyl, pyroglutamyl, seryl, cystyl, threonyl, tyrosyl, tryptophanyl, or valyl.

In other examples, the didpetide can comprise tyrosyl with alanyl, allosoleucyl, arginyl, asparagyl, aspartyl, glutamyl, glutaminyl, glycyl, histidyl, isolelucyl, leucyl, lysyl, methionyl, phenylalanyl, prolyl, pyroglutamyl, seryl, cystyl, threonyl, tryptophanyl, or valyl.

In other examples, the didpetide can comprise methionyl with alanyl, allosoleucyl, arginyl, asparagyl, aspartyl, glutamyl, glutaminyl, glycyl, histidyl, isolelucyl, leucyl, lysyl, phenylalanyl, prolyl, pyroglutamyl, seryl, cystyl threonyl, tyrosyl, tryptophanyl, or valyl.

In other examples, the didpetide can comprise tryptophanyl with alanyl, allosoleucyl, arginyl, asparagyl, aspartyl, glutamyl, glutaminyl, glycyl, histidyl, isolelucyl, leucyl, lysyl, phenylalanyl, prolyl, pyroglutamyl, seryl, cystyl threonyl, tyrosyl, or valyl.

A preferred dipeptide is lysyl-lysyl (KK).

The disclosed conjugate can also comprise three amino acid residues, a tripeptide, linked to the hydrophobic compound. Suitable tripeptides include Xaa-Xbb-Xbb, Xbb-Xaa-Xbb, or Xbb-Xbb-Xaa, where Xaa is arginyl, histidyl, lysyl, aspartyl, glutamyl, seryl, threonyl, cystyl, asparagyl, glutaminyl, prolyl, tyrosyl, methionyl, and tryptophanyl; and wherein each Xbb is independent of the others; alanyl, allosoleucyl, arginyl asparagyl, aspartyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

The disclosed conjugate can also comprise four amino acid residues, a tetrapeptide, linked to the hydrophobic compound. Suitable tetrapeptides include Xaa-Xaa-Xbb-Xbb (SEQ ID NO:1), Xaa-Xbb-Xaa-Xbb (SEQ ID NO:2), Xbb-Xbb-Xaa-Xaa (SEQ ID NO:3), or Xbb-Xaa-Xbb-Xaa (SEQ ID NO:4), where each Xaa is independent of the other, arginyl, histidyl, lysyl, aspartyl, glutamyl, seryl, threonyl, cystyl, asparagyl, glutaminyl, prolyl, tyrosyl, methionyl, and tryptophanyl; and wherein each Xbb is independent of the others, alanyl, allosoleucyl, arginyl asparagyl, aspartyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

In still other examples the conjugate can also comprise five amino acid residues (i.e., a pentapeptide), six amino acid residues (a hexapeptide), seven amino acid residues (a heptapetide), or eight amino acid residue (an octopeptide). In these examples, the peptide has at least three amino acid residues selected from the group consisting of arginyl, histidyl, lysyl, aspartyl, glutamyl, seryl, threonyl, cystyl, asparagyl, glutaminyl, prolyl, tyrosyl, methionyl, and tryptophanyl.

In many examples herein the conjugate does not contain nine or more amino acid residues.

In each example of the disclosed conjugates, the hydrophobic compound can be linked to the peptide at the side chain of one of the amino acid residues. Further, the peptide component can be functionalized, at one or more side chains or at the C or N terminus. For example, the N terminus of the peptide or amino group on a side chain can be protected with a benzoyloxycarbonyl groups, tert-butoxycarbonyl groups, acetate, trifluoroacetate, 9-fluorenylmethyloxycarbonyl, or 2-bromobenzyloxycarbonyl, or N-hydroxysuccinimide, In further examples, the C terminus or relevant side chain can be protected with a methyl, ethyl, t-butyl, or benzyl ester. In a preferred example, the N terminus of the peptide is protected with a 9-fluorenylmethyloxycarbonyl.

### Linker (L)

As noted herein, the disclosed conjugate comprises a hydrophobic compound linked to a single amino acid residue or an amino acid residue of a peptide via a linker moiety. The linker moiety is shown as L in Formula I. The linker moiety of the disclosed conjugates can arise from any compound (linker) that forms a bond with the hydrophobic compound and an amino acid residue, linking them together. Thus, a linker typically contains at least two functional groups, e.g., one functional group that can be used to form a bond with the hydrophobic compound and another functional group that can be used to form a bond with an amino acid residue. Typically, though not necessarily, the functional group on the linker that is used to form a bond with the hydrophobic group is at one end of the linker and the functional group that is used to form a bond with the amino acid is at the other end of the linker.

In some aspects, the linker can comprise electrophilic functional groups that can react with nucleophilic functional groups like hydroxyl, thiol, carboxylate, amino, or amide groups on the hydrophobic compound, forming a bond. Conversely, the linker can comprise nucleophilic functional groups that can react with electrophilic functional groups like carbonyl, halide, or alkoxyl groups on the hydrophobic compound.

The linker can also have one or more electrophilic groups that can react with and thus form a bond to an amino acid residue.

These bonds can be formed by reaction methods known in the art. For example, the hydrophobic compound can be first attached to the linker, followed by attaching the amino acid residue. Alternatively, the linker can be first attached to the amino acid residue and then attached to the hydrophobic compound. Still further, the hydrophobic compound and amino acid residue can both be attached to the linker simultaneously.

The resulting bond between the linker and the hydrophobic compound and amino acid residue should be biodegradable. In this way the compound can be released to the individual and act in its intended way. As such, the bond between the compound and linker, and the bond between the linker and the amino acid residue should be an ester, ether, or amide bond. In many examples herein, the linker moiety does not contain a disulfide bond.

The linker moiety can be of varying lengths, such as from 1 to 20 atoms in length. For example, the linker moiety can be from 1, 2, 3, 4, 5, 6, 7, 8, 9, 10, 11, 12, 13, 14, 15, 16, 17, 18, 19, or 20 atoms in length, where any of the stated values can form an upper and/or lower end point of a range. Further, the linker moiety can be substituted or unsubstituted. When substituted, the linker can contain substituents attached to the backbone of the linker or substituents embedded in the backbone of the linker. For example, an amine substituted linker moiety can contain an amine group attached to the backbone of the linker or a nitrogen in the backbone of the linker.

Suitable linker moieties include, but are not limited to, substituted or unsubstituted, branched or unbranched, alkyl, alkenyl, or alkynyl groups, ethers, esters, polyethers, polyesters, polyalkylenes, polyamines, heteroatom substituted alkyl, alkenyl, or alkynyl groups, cycloalkyl groups, cycloalkenyl groups, heterocycloalkyl groups, heterocycloalkenyl groups, and the like, and derivatives thereof, where the point of attachment to the hydrophobic compound and/or amino acid is an ester, ether, carboxylate, amine, or amide bond.

In one aspect, the linker moiety can comprise a C₁-C₆ branched or straight-chain alkyl, such as methyl, ethyl, n-propyl, iso-propyl, n-butyl, iso-butyl, sec-butyl, tert-butyl, n-pentyl, iso-pentyl, neopentyl, or hexyl. In a specific example, the linker moiety can comprise —(CH₂)ₘ—, wherein m is from 1 to 10, and where the point of attachment to the hydrophobic compound and/or amino acid is an ester, ether, carboxylate, amine, or amide bond. For example, the linker moiety can be X¹—(CH₂)ₘ—X², wherein m is from 1 to 10, and X¹ and X² are, independent of the other, C(O), C(O)O, C(O)N, NH, or O.

In still another aspect, the linker moiety can comprise a C₂-C₆ branched or straight-chain alkyl, wherein one or more of the carbon atoms is substituted with oxygen (e.g., an ether) or an amino group. For example, suitable linkers can include, but are not limited to, a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, methoxymethoxymethyl, ethoxymethoxymethyl, methoxyethoxymethyl, methoxymethoxyethyl, and the like, and derivatives thereof, where the point of attachment to the hydrophobic compound and/or amino acid is an ester, ether, or amide bond.

In a preferred example, the linker moiety is —C(O)CH₂CH₂C(O)—, i.e., a succinate ester.

### Enzymes

Also disclosed are compositions that contain one or more enzymes in the nanotubes.

For example, methane monoxygenase (MMO) can be used for conversion of methane to methanol. This enzyme also has a broad substrate specificity and can be used to make many other useful compounds. Nitrogenases can also be used. In addition to its normal nitrogen fixation reaction, catalyzes the 8 electron transfer reaction by which CO₂ can be reduced to methanol. In addition, nitrogenase can also catalyze the reduction of CO₂ coupled to acetylene to form propylene, an industrially important compound. Methanol dehydrogenase can be used in the first step to convert methanol into a variety of compounds. Pyruvate synthase/pyruvate ferredoxin oxidoreducatase (PS/PFOR) from another CO₂ fixation pathway [the reductive tricarboxylic acid (RTCA) pathway] can be used to produce pyruvate from CO₂ and acetyl-CoA, with pyruvate subsequently converted to additional products with several different enzyme systems. α-ketoglutarate synthase/α-ketogutarate/ferredoxin oxidoreductase (KGS/KGOR) from the RTCA pathway catalyzes the formation of α-ketoglutarate from CO₂ and succinyl-CoA, with α-ketoglutarate converted to several different products with many different enzyme systems.

RubisCO is used to catalyze the reduction of CO₂ to 3-phosphoglyceric acid (3-PGA) as previously noted. Other enzymes of the Calvin-Benson-Bassham (CBB) pathway (3-PGA kinase, 3-phosphoglyceraldedyhe dehydrogenase, triose phosphate isomerase) may be used to convert 3-PGA to dihydroxyacetone phosphate (DHAP). DHAP may be converted to 3-phosphoglycerol (3GP) via 3GP dehydydrogenase. Using a phosphatase enzyme, 3GP can be converted to glycerol, which can be used as a precursor for many chemical syntheses for valuable products.

Production of RubisCO substrate (ribulose 1, 5-bisphosphae, RuBP) from glucose via the use of glucokinase, glucose-6-phosphahe dehydrogenase, phosphoguconate dehydrogenase and phosphoribulokinase.

Production of acrylic acid from CO₂ and acetyl-CoA using acetyl-CoA carboxylase, malyl-CoA reductase, and a trifunctional 3-hydroxypropionate CoA ligase/enoyl-CoA hydratase/enoyl-CoA reductase enzyme, with the CoA reductase region removed so that the enzyme catalyzes the formation of acryl-CoA. Then a CoA transferase is added so the acryl-CoA is converted to acrylic acid. Acrylic acid is a compound that has much industrial interest.

Production of butanol from pyruvate using PS/PFOR, β-ketothiolase, 3-hydroxybutyryl-CoA dehydrogenase, crotonase, trans-2-enoyl-CoA reductase, and aldehyde/alcohol dehydrogenase. Butanol is an excellent biofuel and is used in many industrial applications.

### Specific Examples

In certain aspects, disclosed are compositions that comprise a self-assembled nanotube comprising a conjugate comprising hydrophobic compound, a hydrophilic amino acid residue or peptide; and an optional linker moiety joining the hydrophobic compound to the hydrophilic amino acid or peptide, wherein the conjugate forms a self-assembled nanotube, and an enzyme, wherein the enzyme is sequestered in the self-assembled nanotube. In specific examples, the enzyme is RubisCO. In specific examples, the hydrophobic compound is benzo[lmn][3,8]phenanthroline-1,3,6,8(2H,7H)-tetraone (NDI). In specific examples, the hydrophobic compound is camptothecin. In specific examples, the hydrophilic peptide has from 2 to 9 amino acid residues. In specific examples, the hydrophilic peptide is a dipeptide comprising two protected or unprotected lysine residues. In specific examples, the hydrophilic peptide is a tripeptide comprising at least two protected or unprotected lysine residues. In specific examples, the hydrophilic peptide is a tripeptide comprising one or more of the following hydrophilic amino acid residues protected or unprotected arginyl, histidyl, lysyl, aspartyl, glutamyl, seryl, threonyl, cystyl, asparagyl, glutaminyl, prolyl, tyrosyl, methionyl, and or tryptophanyl. In specific examples, the hydrophilic peptide is a tetrapeptide comprising at least two protected or unprotected lysine residues. In specific examples, the hydrophilic peptide is a tetrapeptide comprising the formula Xaa-Xaa-Xbb-Xbb (SEQ ID NO: 1), Xaa-Xbb-Xaa-Xbb (SEQ ID NO:2), Xbb-Xbb-Xaa-Xaa (SEQ ID NO:3), or Xbb-Xaa-Xbb-Xaa (SEQ ID NO:4) where each Xaa is independent of the other, a hydrophilic amino acid residue chosen from a protected or unprotected arginyl, histidyl, lysyl, aspartyl, glutamyl, seryl, threonyl, cystyl, asparagyl, glutaminyl, prolyl, tyrosyl, methionyl, and tryptophanyl; and wherein each Xbb is, independent of the others, a non-hydrophilic amino acid chosen from protected or unprotected alanyl, allosoleucyl, arginyl asparagyl, aspartyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl. In specific examples, the hydrophilic amino acid or peptide is protected at an N terminus or an amino acid residue side chain with a benzoyloxycarbonyl, tert-butoxycarbonyl, acetate, trifluoroacetate, 9-fluorenylmethyloxycarbonyl, or 2-bromobenzyloxycarbonyl, or N-hydroxysuccinimide. In specific examples, the hydrophobic compound is joined to the hydrophilic amino acid residue or peptide at a side chain on the hydrophilic amino acid or peptide. In specific examples, the hydrophobic compound is joined to the hydrophilic amino acid residue or peptide by the linker, which is attached to the hydrophobic compound and a side chain on the hydrophilic amino acid or peptide. In specific examples, the linker moiety is from 1 to 20 atoms in length. In specific examples, the linker moiety is substituted or unsubstituted, branched or unbranched, alkyl, alkenyl, alkynyl, ether, ester, polyether, polyester, polyalkylene, polyamine, heteroatom substituted alkyl, alkenyl, or alkynyl group, cycloalkyl, cycloalkenyl, heterocycloalkyl, heterocycloalkenyl, where the point of attachment to the hydrophobic drug and/or amino acid residue is an ester, ether, carboxylate, amine, or amide bond. In specific examples, the linker moiety comprises —(CH₂)ₘ—, wherein m is from 1 to 10, and where the point of attachment to the hydrophobic drug and/or amino acid is an ester, ether, carboxylate, amine, or amide bond. In specific examples, the linker moiety comprises —X₁—(CH₂)ₘ—X₂—, wherein m is from 1 to 10, and X₁ and X₂ are, independent of one another, C(═O), C(═O)O, C(═O)NH, NH, or O. In other examples, the peptide is protected or unprotected lysyl-lysyl, or protected or unprotected lysyl-phenylalanyl-lysyl-lysyl, and the linker moiety is C₁-C₆ alkyldiester. In specific examples, the composition further comprising carbonic anhydrase. In specific examples, the conjugate forms the self-assembled nanotube at 10 mM in water.

### Methods of Use

The disclosed materials capitalize on biological and chemical platforms to create a viable, stable catalytic system to convert CO₂ to useful products, directly usable (unlike formate or methanol which would require further chemical processing to be useful by the industry). One example of a product, but not the only product, is acrylic acid from CO₂. Production of acrylic acid largely depends on fossil fuel resources; therefore, due to the rising price of crude oil globally, manufacturers are now focusing on developing and commercializing renewable acrylic acid. The global acrylic acid market is forecast to reach $18.8 billion by 2020 from $11.0 billion in 2013, registering a CAGR (Compound Annual Growth Rate) of 7.6% during the forecast period (2014-2020). Currently no technology exists for production of acrylic acid or lactic acid directly from CO₂. This represents only one downstream application of CO₂ fixation. However, many other small feedstock materials such as butanol, butadiene and others may be possible with this technology.

Biology-inspired catalysts derived from bacteria and plants, such as ribulose-1,5-bisphosphate carboxylase oxygenase (RubisCO), efficiently extract CO₂ from air and convert it to energy-rich compounds like glucose; these conversions entail the sequential action of multiple enzymes. In addition, other key catalysts such as nitrogenase and methane monooxygenase (MMO) are also known to catalyze key greenhouse gas conversions. Nitrogenase, in addition to its well-known ability to catalyze N₂ reduction is also able to catalyze the 8-electron reduction of CO₂ to CH₄, and also other products. MMO catalyzes the oxidation of CH₄ to CH₃OH, an important industrial product for further synthetic processes. However, the use of free or cell-based enzymes as biocatalysts for large-scale industrial processes pose significant drawbacks due to their incompatibility with reaction conditions that often depart from their physiological states. The challenge is to construct catalytic systems that mimic the cellular environment but are (i) scalable, (ii) robust to withstand harsher conditions, and (iii) amenable to further development into devices that may be strategically deployed at sources/repositories of these greenhouse gases. Furthermore, cells often compartmentalize various biological reactions to address challenges such as the toxicity of accumulating intermediates, competing reaction pathways and slow turnover rates. The disclosed materials focus on mimicking biological compartmentalization, such as in carboxysomes, structures that naturally encapsulate RubisCO and carbonic anhydrase, by co-encapsulating catalytic systems with CO₂/CH₄ concentrating materials and photosynthetic energy sources. The capsules described in this application are synthetic nanostructured capsules, such as nanotubes, nanofibers or nanoribbons in order to enhance catalytic activity and stability.

Disclosed are scalable catalytic systems that can be deployed at repositories of greenhouse gases for converting them to useful products. Biological catalysts function by reducing the energy required to bring reactants together for product formation and often operate optimally at physiological ionic conditions and temperatures. Furthermore, biocatalysts are highly specific, making them convenient and desirable vehicles for combining a series of steps, all under one roof, leading to a specific product. The use of a cell-free catalytic system can be advantageous because it allows for deployment at harsher conditions that are typical of greenhouse-gas repositories. Further, the use of biological hosts poses challenges in the form of media requirements, maintaining a contamination-free environment, dealing with side products and the requirement to frequently replenish the cell material.

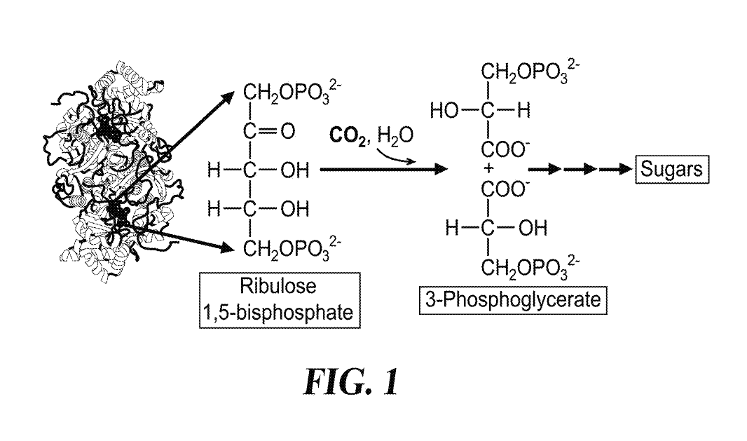

As the world's most abundant enzyme, which accounts for most of the carbon flux sustaining life on this plant, RubisCO has been well studied and is an attractive target for catalyzing the first step of CO₂ capture from the greenhouse gases as part of various biotechnological applications. It catalyzes the reduction and assimilation of CO₂ onto a 5-carbon compound, ribulose 1,5-bisphoshphate (RuBP), resulting in the formation of two 3-carbon (3-phosphoglycerate) molecules (FIG. 1). Several structural variants of RubisCO with varying structural complexity and catalytic properties can be used, ranging from the structurally simple enzyme from bacteria (dimer of two identical catalytic subunits) to the more complex 16-subunit enzyme from bacteria, algae and plants, containing 8 large and 8 small subunits.

Because RubisCO is the primary step for CO₂ capture and is often the rate-limiting step, RubisCO was used as a model protein for encapsulation in macromolecular scaffolds such as organic nanotubes and electro polymers. To this end, RubisCO has been successfully encapsulated within nanotubes. Functionality has been demonstrated for the encapsulated enzymes and preliminary results clearly indicate that these scaffolds impart better stability and/or resilience to the enzyme in comparison to the free form. The experimental details and results obtained are outlined.

In specific examples, disclosed herein are a methods of catalyzing the conversion of CO₂ into an organic compound comprising; contacting a composition disclosed herein with CO₂. The CO₂ can be in air or in a flue or industrial gas.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Self-Assembly of Nanotubes and Interaction with RubisCO

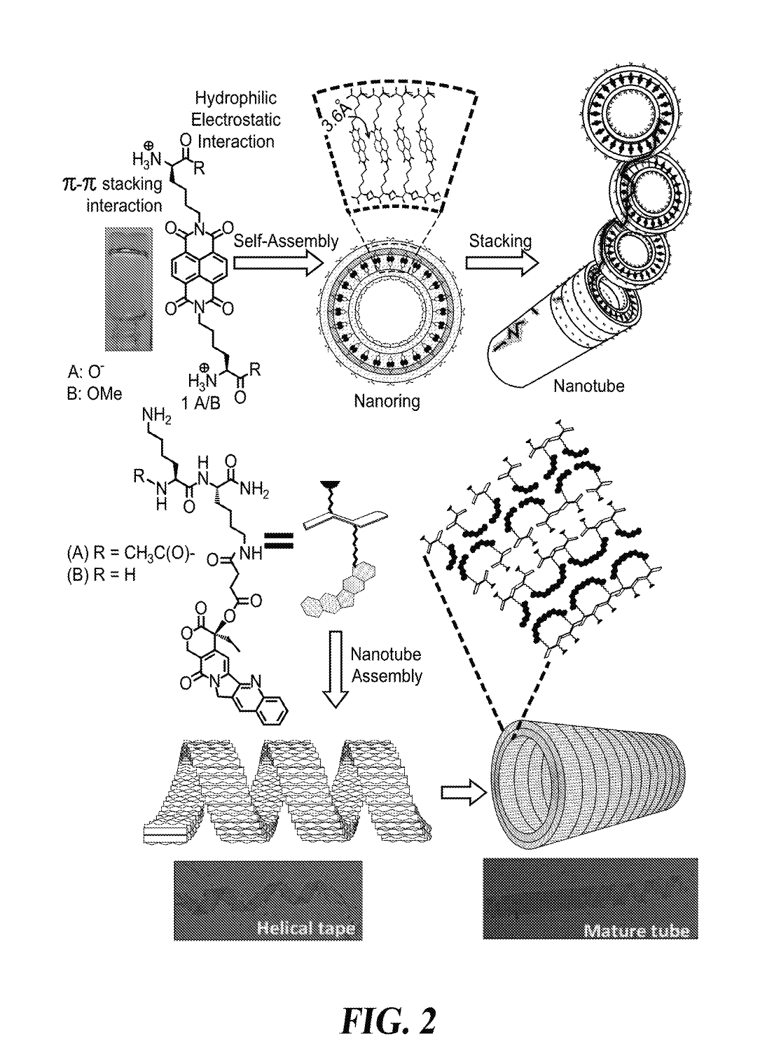

Simple lysine-NDI conjugates (FIG. 2) undergo self-assembly into nanotubes ranging in diameter from 14-18 nm to 200 nm in water. In these structures, the NDI chromophore (naphthalene diimide or benzo[lmn][3,8]phenanthroline-1,3,6,8(2H,7H)-tetraone) serves as a nonpolar tail capable of hydrophobic π-π association, whereas lysine provides both the polar headgroup and molecular chirality of the amphiphile. Self-assembly proceeds via a bilayer membrane followed by the formation of twisted ribbons, which then transform into coiled ribbons. Based on the bilayer model of the assembly for the NDI-lysine amphiphiles, the assembly of nanotubes in water using a single, boloamphiphilic version of the bilayer is realized (FIG. 2, top). In contrast to the amphiphile shown in FIG. 2, bolaamphiphilic assembly proceeds via ring formation followed by stacking of the rings into the nanotubes. The interiors of these (bola)amphiphilic nanotubes are hydrophilic, water-filled regions with dimensions up to ˜200 nm. The systems are ideally suited to accommodate these proteins within their internal regions.

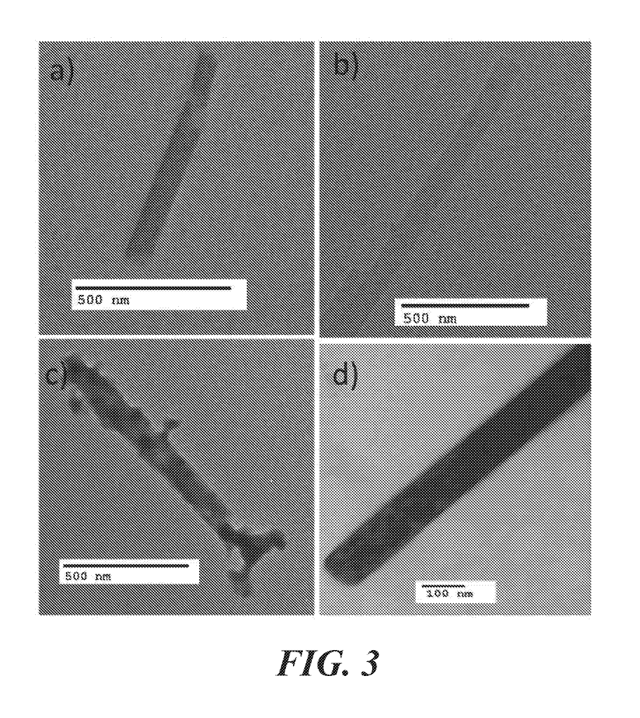

Initial studies whereby a RubisCO dimer from the bacterium Rhodospirillum rubrum was mixed with the nanotubes [1 mM nanotube/RubisCO (0.1 mg·mL, 50 mM, Tris-Cl, 10 mM MgCl₂, pH 7.2)] induced complete precipitation of the nanotubes. Each of the nanotubes shown in FIG. 2 were studied by mixing with RubisCO at a range of concentrations, all of which resulted in precipitated proteins. To address the incompatibility of the nanotubes with RubisCO, a series of dipeptide-camptothecin (CPT) nanotubes were used. This series of nanotubes were designed to self-assemble into nanotubes in PBS and serum by using lysine residues to position charged ammonium groups on the surface of the nanotubes. Such an approach is expected to enhance solubility and attenuate aggregation caused by electrostatic repulsion of the ammonium groups. Part of the incompatibility of the nanotubes arises from the screening of charge by the buffer which leads to precipitation of the nanotubes in the buffered systems necessary for RubisCO. Thus, the CPT-dipeptides, Ac-KK-CPT (A) and NH₂-KK-CPT (B), shown in FIG. 2 were used. These dipeptides were exceptionally compatible with the Tris-Cl-buffered conditions and RubisCO, resulting in strong encapsulation of RubisCO. The low contrast between RubisCO and the nanotubes made visualization the RubisCO/nanotube complex difficult to resolve, but binding of the enzyme by the nanotube was apparent in the images when stain was not used (FIG. 3A-3D). However, centrifugation of the solutions at 27000 g for 60 min at 4° C., conditions that do not pellet RubisCO alone, pelleted all of the enzyme, as discussed herein.

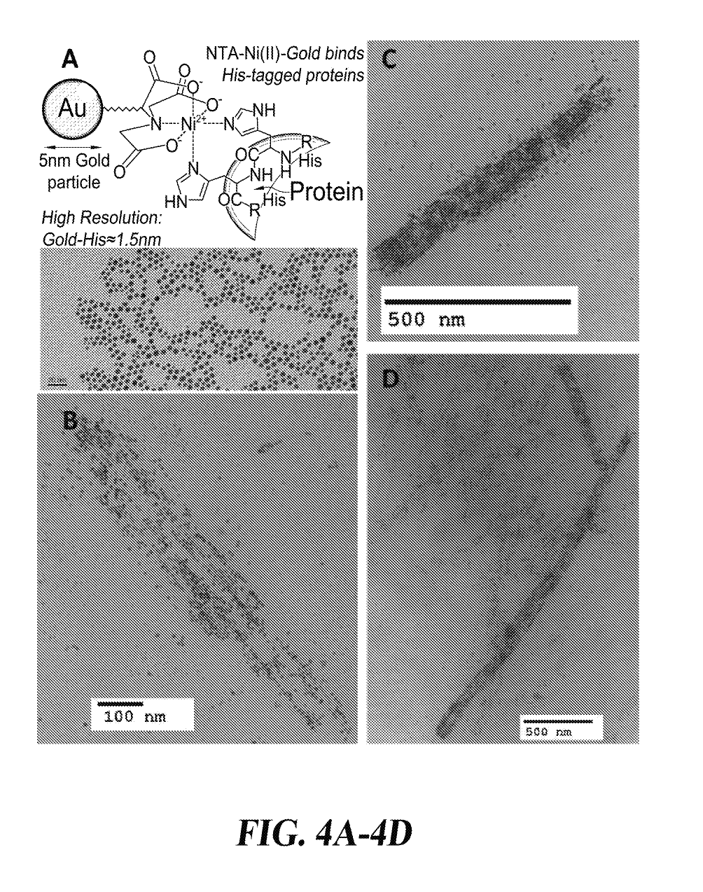

To enhance the contrast between the nanotubes and the enzyme during TEM imaging, complexed Ni-NTA-NANOGOLD™ particles were complexed to a poly-histidine tagged R. rubrum RubioCO prior to binding to the nanotubes (FIG. 4A-4D). As can be observed in FIGS. 4B-4C, large amounts of the Nanogold-tagged RubisCO could be observed as black dots along the inner and outer surface of the nanotubes.

Results of the RubisCO are summarized below:
10-25% of starting RubisCO activity was recoverable after assembly into nanotubes, depending on the nature and amount of organic monomer used; higher RubisCO concentrations (>0.1 mg/ml) causes the protein to precipitate out
No RubisCO was detectable in supernatants after centrifugation steps, indicating that all added RubisCO was associated with the nanotube pellets.
All nanotube preparations tested thus far appear to protect RubisCO from proteolysis (67-100% recovery) but not from heat-induced denaturation.
Results from initial RubisCO functional assays:

| | | Activity | % |
|---|---|---|---|
| | | (nmoles/ | Activity |
| Sample | Treatment | min/mg) | retained |
| Pure R. rubrum RubisCO | | 2903 | 100 |
| 0.1 mg/ml R. rubrum | Untreated | 3007 | 104 |
| RubisCO | Heat (65° C./10 min) | 2248 | 77 |
| (after 80K spin) | Subtilisin (0.5 μg/ml; | 212 | 7 |
| | 30° C./1 hr) | | |
| 5 mM NH2-KK-CPT + | Nanotube only | ~0 | 0 |
| 0.1 mg/ml R. rubrum | Untreated | 289 | 10 |
| RubisCO | Heat (65° C./10 min) | 75 | 3 |
| | Subtilisin (0.5 μg/ml; | 283 | 10 |
| | 30° C./1 hr) | | |
| 5 mM Ac-KK-CPT + | Nanotube only | ~0 | 0 |
| 0.1 mg/ml R. rubrum | Untreated | 440 | 15 |
| RubisCO | Heat (65° C./10 min) | 118 | 4 |
| | Subtilisin (0.5 μg/ml; | 293 | 10 |
| | 30° C./1 hr) | | |
| 5 mM NH2-K-CPT + | Nanotube only | ~0 | 0 |
| 0.1 mg/ml R. rubrum | Untreated | 208 | 7 |
| RubisCO | Heat (65° C./10 min) | 9 | 0 |
| | Subtilisin (0.5 μg/ml; | 204 | 7 |
| | 30° C./1 hr) | | |

### Example 2: Enhanced Activity of Nanotube-Bound RubisCO

It was found that different samples of nanotubes derived from dipeptides A and B resulted in widely different catalytic activities upon co-assembly with RubisCO ranging from 1.5% to 35%. Although the samples were identical in purity and composition, the resultant activity of the bound RubisCO varied. After significant study, it was determined that the nanotube precursor (monomers of A or B) inhibited the enzyme. Thus, by pelleting the nanotube by ultracentrifugation, followed by additional washing of the pellet, prior to adding the enzyme, the activity increased dramatically to 67%. Further experiments have revealed that this procedure leads to near native activity for the RubisCO-nanotube hybrid. The table below shows the activity for nanotube, purified by ultracentrifugation, as described above, along with preliminary studies using a tetrapeptide nanofiber-RubisCO coassembly.

### Example 3: Optimizing Activity/Stability of RubisCO within the Nanotubes

Modulating the surface charge (Zeta potential) of the nanotube surface can optimize the interaction between the nanotubes and RubisCO to enhance enzyme activity. This is based on the observation that the nanotubes bind RubisCO very strongly, resulting in no activity observed within the supernatant, and the visualization of RubisCO particles adhered to the inner and outer surface of the nanotubes on the TEM images. The Zeta potential of dipeptides NH₂-KK-CPT and Ac—KK-CPT are +39 and +27, respectively. 42 peptides of 6 combinations of 7 peptides (X) were made: where X=arginine, asparagine, aspartic acid, cysteine, glutamic acid, glutamine, or histidine (Ac-K(CPT)-X—NH₂; NH₂—K(CPT)-X—NH; CPT-K—X—NH₂; Ac—X—K(CPT)-NH₂; NH₂—X—K(CPT)-NH₂; CPT-X—K—NH₂). All the dipeptides, except two of the cysteine containing peptides (due to disulfide bond formation) assembled into nanotubes with diameters ranging from 80-110 nm) and with Zeta potentials ranging from −9 to +39.

RubisCO activity is inhibited by monomers by not pre-formed nanotubes

| | Activity | % Activity |
|---|---|---|
| Sample | (nmoles/min-mg) | retained |
| Pure R. rubrum RubisCO | 3405 | 100 |
| + Nanotubes (from NH₂-KK-CPT) | 3063 | 90 |
| + Nanotubes (from Ac-KK-CPT) | 3022 | 89 |
| +5 nM NH₂-KK-CPT (monomer) | 1396 | 41 |
| +5 nM Ac-KK-CPT (monomer) | 1213 | 36 |

Newer monomer preps were also less soluble

RubisCO activity inhibition by the monomer FMOC-KFKK-Benzene
(forms nanofibers)

| | Activity | % Activity |
|---|---|---|
| Sample | (nmoles/min-mg) | retained |
| Pure R. rubrum RubisCO | 3405 | 100 |
| +5 nM FMOC-KFKK-Benzene (SEQ ID | 1863 | 55 |
| NO: 5, underlined portion only) | | |
| (monomer) | | |

Final nanofibers (obtained as precipitate after spinning preps at 20000 Xg for 10 min) retained only ˜4% activity.

Assembly of RubisCO with “pre-formed” nanotubes gives
maximum recovery of activity

| | Activity | % Activity |
|---|---|---|
| Sample | (nmoles/min-mg) | retained |
| Pure R. rubrum RubisCO | 1803 | 100 |
| Pre-formed nanotubes (NH₂-KK-CPT) + | 1210 | 67 |
| R. rubrum RubisCO | | |
| Pre-formed nanotubes (NH2-KK-CPT)- | 0 | 0 |
| Control | | |
| Supernatant ultracentrifugation (final | 0 | 0 |
| nanotubes preps) | | |

Nanotubes for this experiment were set up with 10 mM monomer in 1 mL buffer and aged for ˜5 days at room temperature. Nanotubes were then isolated using ultracentrifugation and re-suspended back in 1 mL buffer with 0.1 mg of R. rubrum RubisCO. This suspension was incubated at 1° C. for 15 hrs prior to ultracentrifugation and re-suspension of the final nanotubes in 1 mL buffer.

### Example 4: Stability to Peptidase, Subtilisin

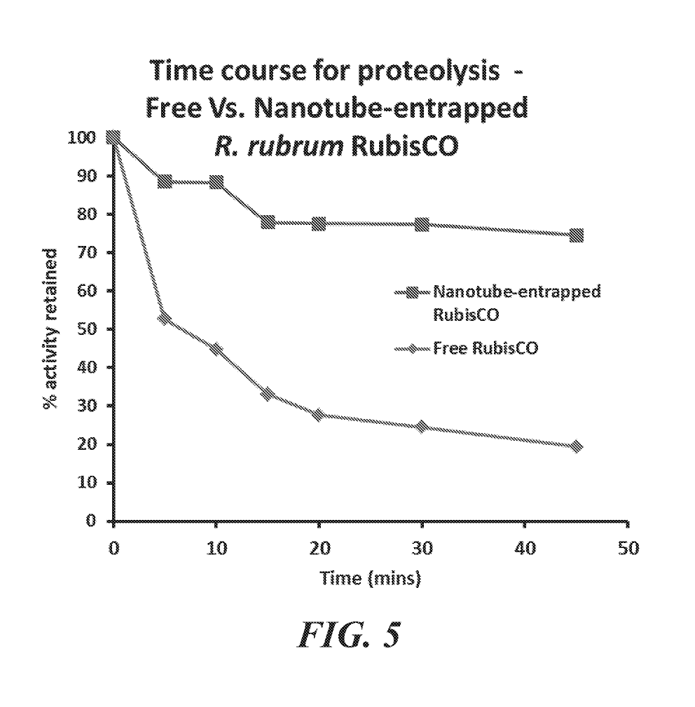

Subtilisin is a non-specific, serine protease capable of rapidly degrading proteins by amide bond cleavage. The stability of the nanotube-RubisCO co-assembly to proteolysis by subtilisin was evaluated over 45 minutes and compared to the free R. rubrum RubisCO. As shown in FIG. 5, the free enzyme loses 80% of the activity within 45 minutes. In contrast, the bound enzyme retains 75% of its activity upon exposure to the protease over this time range.

The materials and methods of the appended claims are not limited in scope by the specific materials and methods described herein, which are intended as illustrations of a few aspects of the claims and any materials and methods that are functionally equivalent are within the scope of this disclosure. Various modifications of the materials and methods in addition to those shown and described herein are intended to fall within the scope of the appended claims. Further, while only certain representative materials, methods, and aspects of these materials and methods are specifically described, other materials and methods and combinations of various features of the materials and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. A composition, comprising: a self-assembled nanotube comprising a conjugate comprising hydrophobic compound, a hydrophilic amino acid residue or peptide; and an optional linker moiety joining the hydrophobic compound to the hydrophilic amino acid or peptide, wherein the conjugate forms a self-assembled nanotube, and an enzyme, wherein the enzyme is sequestered in the self-assembled nanotube.

2. The composition of claim 1, wherein the enzyme is RubisCO.

3. The composition of claim 1, wherein the hydrophobic compound is benzo[lmn][3,8]phenanthroline-1,3,6,8(2H,7H)-tetraone (NDI).

4. The composition of claim 1, wherein the hydrophobic compound is camptothecin.

5. The composition of claim 1, wherein the hydrophilic peptide has from 2 to 9 amino acid residues.

6. The composition of claim 1, wherein the hydrophilic peptide is a dipeptide comprising two protected or unprotected lysine residues.

7. The composition of claim 1, wherein the hydrophilic peptide is a tripeptide comprising at least two protected or unprotected lysine residues.

8. The composition of claim 1, wherein the hydrophilic peptide is a tripeptide comprising one or more of the following hydrophilic amino acid residues protected or unprotected arginyl, histidyl, lysyl, aspartyl, glutamyl, seryl, threonyl, cystyl, asparagyl, glutaminyl, prolyl, tyrosyl, methionyl, and or tryptophanyl.

9. The composition of claim 1, wherein the hydrophilic peptide is a tetrapeptide comprising at least two protected or unprotected lysine residues.

10. The composition of claim 1, wherein the hydrophilic peptide is a tetrapeptide comprising the formula Xaa-Xaa-Xbb-Xbb (SEQ ID NO:1), Xaa-Xbb-Xaa-Xbb (SEQ ID NO:2), Xbb-Xbb-Xaa-Xaa (SEQ ID NO:3), or Xbb-Xaa-Xbb-Xaa (SEQ ID NO:4), where each Xaa is independent of the other, a hydrophilic amino acid residue chosen from a protected or unprotected arginyl, histidyl, lysyl, aspartyl, glutamyl, seryl, threonyl, cystyl, asparagyl, glutaminyl, prolyl, tyrosyl, methionyl, and tryptophanyl; and wherein each Xbb is, independent of the others, a non-hydrophilic amino acid chosen from protected or unprotected alanyl, allosoleucyl, arginyl asparagyl, aspartyl, cystyl, glutamyl, glutaminyl, glycyl, histidyl, isolelucyl, leucyl, lysyl, methionyl, phenylalanyl, prolyl, pyroglutamyl, seryl, threonyl, tyrosyl, tryptophanyl, or valyl.

11. The composition of claim 1, wherein the hydrophilic amino acid or peptide is protected at an N terminus or an amino acid residue side chain with a benzoyloxycarbonyl, tert-butoxycarbonyl, acetate, trifluoroacetate, 9-fluorenylmethyloxycarbonyl, or 2-bromobenzyloxycarbonyl, or N-hydroxysuccinimide.

12. The composition of claim 1, wherein the hydrophobic compound is joined to the hydrophilic amino acid residue or peptide at a side chain on the hydrophilic amino acid or peptide.

13. The composition of claim 1, wherein the hydrophobic compound is joined to the hydrophilic amino acid residue or peptide by the linker, which is attached to the hydrophobic compound and a side chain on the hydrophilic amino acid or peptide.

14. The composition of claim 1, wherein the linker moiety is from 1 to 20 atoms in length.

15. The composition of claim 1, wherein the linker moiety is substituted or unsubstituted, branched or unbranched, alkyl, alkenyl, alkynyl, ether, ester, polyether, polyester, polyalkylene, polyamine, heteroatom substituted alkyl, alkenyl, or alkynyl group, cycloalkyl, cycloalkenyl, heterocycloalkyl, heterocycloalkenyl, where the point of attachment to the hydrophobic drug and/or amino acid residue is an ester, ether, carboxylate, amine, or amide bond.

16. The composition of claim 1, wherein the linker moiety comprises —(CH₂)ₘ—, wherein m is from 1 to 10, and where the point of attachment to the hydrophobic drug and/or amino acid is an ester, ether, carboxylate, amine, or amide bond.

17. The composition of claim 1, wherein the linker moiety comprises —X₁—(CH₂)ₘ—X₂—, wherein m is from 1 to 10, and X₁ and X₂ are, independent of one another, C(═O), C(═O)O, C(═O)NH, NH, or O.

18. The composition of claim 1, wherein the peptide is protected or unprotected lysyl-lysyl, or protected or unprotected lysyl-phenylalanyl-lysyl-lysyl, and the linker moiety is C₁-C₆ alkyldiester.

19. The composition of claim 1, further comprising carbonic anhydrase.

20. The composition of claim 1, wherein the conjugate forms the self-assembled nanotube at 10 mM in water.

21-23. (canceled)
